(19) United States
(12) Patent Application Publication
(10) Pub. No.: US 2002/0026228 A1
(43) Pub. Date: Feb. 28, 2002

(54) Electrode for intravascular stimulation, cardioversion and/or defibrillation

(76) Inventor: Patrick Schauerte, Herzogenrath (DE)

Correspondence Address: Stephen L. Grant, Oldham & Oldham Co., L.P.A., Twin Oaks Estate, 1225 West Market Street, Akron, OH, 44313-7188, US

(21) Appl. No.: 09/726,499

(22) Filed: Nov. 30, 2000

(30) Foreign Application Priority Data
Nov. 30, 1999 (DE) 199 57 662.9

(51) Int. Cl. A61N 1/05

(52) U.S. Cl. 607/122

(57) ABSTRACT

An electrode for intravascular stimulation, cardioversion and/or defibrillation in the form of a stimulation probe which can be fixed in arterial/venous vessels of the body and by way of which electrical or magnetic pulses and defibrillation/cardioversion shocks can be delivered, which is provided with a feed line, wherein there is provided a metallic, electrically conductive, tubular wire unit which adjoins the feed line in the axial direction and which forms an expansion body and which can be deployed in the corresponding vessel and which under expansion bears from the interior against the wall of the vessel.

The invention concerns an electrode for intravascular stimulation, cardioversion and/or defibrillation in the form of a stimulation probe which can be fixed in arterial/venous vessels of the body and by way of which electrical or magnetic pulses and defibrillation/cardioversion shocks can be delivered.

### BACKGROUND OF THE ART

The electrical stimulation of biological tissues is a wide-spread therapeutic principle. Thus atrial and/or ventricular myocardial electrical stimulation of the heart is used in bradycardial (slow) and tachycardial (fast) cardiac disrhythmia situations (cardiac pacemaker and atrial/ventricular cardioverter/defibrillators respectively). Electrical stimulation of the gastro-intestinal tract and the bladder is also used in situations involving motility disturbances to the gastrointestinal tract and the bladder and also in relation to replacement stomachs/replacement intestines/replacement bladders provided by an operative procedure. The electrical stimulation of nerves can be therapeutically used in pain therapy (referred to as TENS devices) and can also be employed for therapy purposes in relation to convulsions (epileptic attacks). Electrical stimulation of the cardial autonomous nervous system for controlling the heart rate in the event of tachycardial supraventricular disrhythmias is also known.

In order to be able to electrically stimulate those biological tissues stimulation electrodes are generally operatively anchored on or in the tissue in question. Particularly in the context of autonomous nerve stimulation, operative fixing of stimulation electrodes on the nerves in question is difficult as those nerves frequently run along blood vessels which can be damaged in the electrode placement procedure and in addition the formation of scar tissue around the nerve stimulation electrode can easily occur post-operatively. This latter phenomenon can result in an increase in the stimulation threshold to the level of loss of stimulation. Electrodes which are arranged within the blood vessels and which float in the circulatory system are also not suitable for nerve stimulation purposes.

In the case of cardiac stimulation, for that purpose probes which are advanced into the heart by way of blood vessels are anchored in the heart muscle tissue. The predominant procedure involves those probes being advanced transveneously after puncturing/incision of the vena cephalica/vena subclavia by way of the superior vena cava into the right atrium or into the right ventricle and anchored there. In recent times electrical stimulation of the left atrium (for example to prevent atrial fibrillation) or the left ventricle (in the event of cardiac insufficiency) has also been implemented in special clinical studies. For that purpose the stimulation electrodes are positioned either epicardially or transveneously by way of the coronary vein sinus in the region of the left atrium/ventricle. Fixing stimulation electrodes in the coronary sinus is technically demanding and partial occlusion of the coronary sinus branches by the stimulation probe can occur.

U.S. Pat. No. 5,954,761 discloses a stimulation electrode having a fixing unit in the form of a stent. In that arrangement a conductor extends in the interior of the stent.

A disadvantage with that arrangement is that the cross-section of the part of the conductor which extends in the interior of the stent reduces the region available for the flow of blood.

In consideration thereof, the object of the invention is to provide an electrode, in particular a nerve stimulation electrode, which can be anchored in a blood vessel without resulting in a substantial reduction in the flow of blood in that vessel.

### SUMMARY OF THE INVENTION

In accordance with the invention the electrode comprises an electrically conductive metal tubular wire item which—corresponding to a stent —is deployed in the corresponding vessel and bears from the interior against the vessel wall. The electrically conductive wire item joins the conductor of the electrical feed line in the axial direction. Accordingly, within the wire item, there is no need for a further line which could reduce the part of the vessel available for the flow of blood. In that respect what is particularly advantageous in the case of the invention is the fact that a balloon which is possibly provided within the wire item for expansion thereof is also not impeded by a feed line extending there.

In that arrangement the electrically conductive wire item can be in the form of a coil or mesh or also in the form of an expandable cylindrical body having openings.

Deployment of the wire item forming an expansion body can be effected actively by means of a balloon which can be inflated pneumatically or by liquid and which is disposed in the non-deployed wire item.

Alternatively, passive deployment (self-expansion) of the wire item can also occur after removal of a compression sleeve.

The metallic, electrically conductive material is elastically or plastically deformable, depending on whether this involves a self-expanding stent or a stent which can be expanded by a balloon.

The entire surface of the wire item or one or more parts of the wire item, which are electrically insulated from each other, can be used as a stimulation pole (unipolar/bipolar/multipolar). In particular the electrodes can be of an elongate configuration corresponding to the course of a nerve (see DE 197 58 114 A1).

The wire item or electrically insulated parts of the wire item are connected with electrically conductive cables to a stimulation unit. It is possible to provide for unipolar/bipolar/multipolar stimulation by way of the entire surface of the wire item or mutually electrically insulated parts thereof. Bipolar stimulation between the wire item/parts of the wire item and a further conventional stimulation probe implanted in the proximity of the wire item or a further wire item probe is also possible. If a plurality of parts of the wire item, which occur in succession in the axial direction, are electrically connected to a feed line, then the feed line in the region of the wire item does not extend within same but is woven into the wire item (in insulated relationship) or in some other fashion forms an electrically insulated part of the wire item which extends independently within the cylindrical shape in the longitudinal direction.

Another preferred embodiment of the electrode according to the invention provides for use in the form of an implantable Helmholtz coil, by way of which an alternating magnetic field can be applied for nerve stimulation purposes.

A further version of the wire unit forming an expansion body comprises a conductive wire which is wound circularly in the manner of a coil and which comprises a fixed number of turns. The wire item can comprise one or more electrically mutually insulated coils. Each of those coils can be electrically connected to a cable which has a connection to an electrical stimulation unit.

For the avoidance of re-stenoses it can also be provided that the electrode is inductively heated from the exterior.

The coil stent can however also be used as an electrode, in conjunction with an induction unit which is implanted in the proximity or which is external and applied to the surface of the body on the outside and which does not have any direct electrical connection to the wire item. That induction unit produces inductively by way of an alternating magnetic or electrical field in the coil stent a voltage field or a magnetic field which can be used for the stimulation of biological tissues such as nerves or musculature.

The diameter and the length of the wire unit forming an expansion body depends on the diameter, the length and the curvature of the blood vessel in which the wire unit forming an expansion body is to be implanted. The diameter of the deployed wire item can be constant or vary over the entire length of the wire item. Thus for example a conical wire item can be used for implantation of the wire unit forming an expansion body, in the proximal coronary sinus. That permits continuous wall contact of the wire unit forming an expansion body, in the region of the mouth opening of the coronary sinus, which decreases in a funnel-like configuration. The length of the wire unit forming an expansion body can be a few millimeters (annular shape) or amount to several centimeters. The surface of the wire unit forming an expansion body can also be coated with medicaments which are intended to alleviate damage to the vessel in which the wire unit forming an expansion body is implanted (for example a corticosteroid coating).

Placement of the wire unit forming an expansion body can be implemented transvascularly with or without X-ray examination. For that purpose, the electrode stent including the electrode stem which incorporates the electrical feed line to the wire unit forming an expansion body is advanced into the appropriate target vessel by way of a guide wire. The guide wire is previously positioned in the vessel under X-ray examination or with echocardiographic monitoring. The wire unit forming an expansion body including the electrode stem has a central or eccentric lumen so that the wire unit forming an expansion body can be advanced into the vessel over the guide wire. Alternatively, the wire unit forming an expansion body may also be without a lumen. In that case, only the wire item is advanced by way of a guide wire which has been previously placed in the vessel and the electrode stem slides along but not over the guide wire.

Expansion of the wire unit forming an expansion body in the vessel is effected by an inflatable balloon which is placed in the wire item. Inflation can be effected pneumatically or by liquid. The balloon can be set in place by way of a guide wire disposed in the lumen of the wire unit stem forming an expansion body, or a guide wire which extends outside the electrode stem but through the wire item.

The pulse-production device is an implantable voltage/magnetic field generator which is capable of producing electrical/magnetic stimulation pulses. The pulse duration can be between 0 and 20 ms (typically between 0.05 and 5 ms) and the stimulation frequency can be between 0 and 1000 Hz (typically between 10 and 100 Hz for nerve stimulation and between 0.5 and 3 Hz for myocardial stimulation). The pulse shape can be monophase, biphase or triphase. Another variant of the pulse-production device is capable of producing an alternating voltage/magnetic field which induces a magnetic/voltage field in the wire item coil. Such a stimulation unit can be implanted in the proximity of the wire item without having a direct electrical connection to the wire coil. Alternatively it is also possible to use an external stimulation unit which can be applied to the surface of the body on the outside thereof, for producing a voltage/magnetic field. A further variant of the pulse-production unit is capable of delivering high-voltage pulses (defibrillation/cardioversion pulses) by way of the wire unit which forms an expansion body (pulse voltage between 50 and 1000 V, pulse duration between 0.5 and 30 ms and pulse shape mono-/bi-/triphase).

The stimulation unit further comprises a detection unit connected to one or more measurement probes which detect biological measurement parameters such as heart rate, blood pressure, oxygen partial pressure, repolarisation times and changes in the excitation recovery of the heart. A start unit which is responsive to the detection parameters sets the pulse-production unit in operation as soon as the measurement parameter falls below or exceeds a given programmed limit value.

The essence of the described wire unit which forms an expansion body permits different uses.

Stimulation of parasympathetic autonomous nerve fibers for reducing the atrial and ventricular frequency in the case of tachycardial disrhythmia phenomena. For that purpose implantation of the wire unit forming an expansion body can be implemented in the arteria/vena jugularis interna or externa, the superior vena cava, the proximal coronary sinus or the inferior vena cava at the boundary to the right atrium.

Stimulation of autonomous nerve fibers for improving the coronary artery blood supply. For that purpose implantation of the wire unit forming an expansion body can be implemented in the arteria/vena jugularis interna or externa and in the coronary sinus.

Stimulation of sympathetic autonomous nerve fibers for the treatment of arterial hypotonia and heart pumping weakness in a case of acute and chronic heart insufficiency. For that purpose implantation of the wire unit forming an expansion body can be implemented in the arteria/vena subclavia, the pulmonary veins or the aorta.

Stimulation of sympathetic autonomous nerve fibers for the treatment of arterial hypotonia and bradycardia in the case of neuro-cardiogenic syncopes. For that purpose implantation of the wire unit forming an expansion body can be implemented in the arteria/vena subclavia, the pulmonary veins or the aorta.

Stimulation of parasympathetic autonomous nerve fibers for the treatment of tachycardial ventricular disrhythmias. For that purpose implantation of the wire unit forming an expansion body can be implemented in the coronary sinus or the pulmonary artery.

Stimulation of parasympathetic nerves which innervate the atria for preventing an atrial remodelling process. For that purpose implantation of the wire unit forming an expansion body can be implemented in the arteria/vena jugularis interna or externa, the superior vena cava or the right pulmonary artery.

Stimulation of parasympathetic nerves which innervate the atria/ventricles for a reduction in the atrial/ventricular defibrillation threshold. For that purpose implantation of the wire unit forming an expansion body can be implemented in the arteria/vena jugularis interna or external the superior vena cava or the right pulmonary artery.

Stimulation of autonomous parasympathetic nerve fibers for the treatment of cerebral convulsions (epilepsy). For that purpose implantation of the wire unit forming an expansion body can be implemented in the arteria/vena jugularis interna or externa.

Stimulation of the carotid sinus nerves for the treatment of angina pectoris complaints. For that purpose implantation of the wire unit forming an expansion body can be implemented in the arteria/vena jugularis interna or externa.

Stimulation of autonomous nerves which regulate gastrointestinal and bladder motility and control male erection. For that purpose implantation of the wire unit forming an expansion body can be implemented in the inferior vena cava and the feeds thereto, the aorta abdominalis and the outflows therefrom (for example aa. mesentericae) or the arterial and venus iliac vessels.

High-frequency, sub-threshold electrical stimulation of the ventricular myocardium for the promotion of angiogenesis after cardiac infarcts or myocardial blood supply disturbances. For that purpose implantation of wire item electrodes can be implemented in the coronary arteries or the coronary sinus and its feeds.

### BRIEF DESCRIPTION OF THE DRAWINGS

The invention is described in greater detail hereinafter by means of preferred embodiments with reference to the drawing in which:

FIG. 1 shows a first embodiment with a wire unit which is in the form of a cylindrical mesh,

FIG. 2 shows an electrode with a balloon for deployment purposes,

FIG. 3 shows a view corresponding to FIG. 2 with only one electrical connection and a guide wire,

FIG. 4 shows a bipolar embodiment which is divided electrically in the longitudinal direction,

FIG. 5 shows a bipolar embodiment which is divided in the axial direction,

FIG. 6 shows an embodiment for the superior vena cava,

FIG. 7 shows an embodiment for the coronary vein sinus,

FIG. 8 shows a conically enlarged embodiment for the coronary sinus ostium,

FIG. 9 shows an embodiment in the form of a narrow ring,

10 shows an embodiment in coil shape for inductive excitation by an external alternating field,

FIG. 11 shows an embodiment in coil shape for excitation by way of an internal coil on a guide wire, and

FIG. 12 shows an embodiment with a connected stimulation control unit.

### DETAILED DESCRIPTION OF THE INVENTION

In the embodiment illustrated in FIG. 1 the electrode 1 according to the invention includes a cylindrical wire unit 2.1 forming a bipolar reference electrode. The wire unit 2.1 comprises an electrically conductive, metallic wire item which is expandable, in the case of an elastic configuration being self-expandable. The flexible electrode feed line (probe) 5 is terminated with a ring 5a forming a bipolar reference electrode. An electrical connection 3 is provided between the end of the (electrically insulated) feed line and the wire unit 2.1. It will be apparent that the wire unit 2.1 and the feed lines are arranged in succession in the axial direction. The interior of the cylindrical wire unit is completely free so that the flow of blood in the vessel is not impeded.

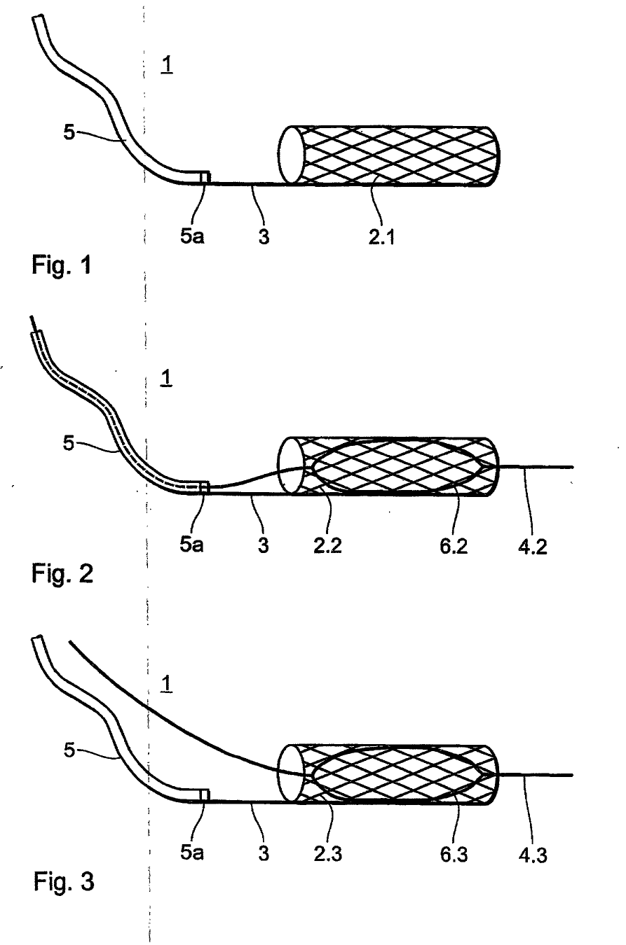

The embodiment shown in FIG. 2 illustrates how the wire unit 2 which in this case is plastically deformable is guided over a guide wire 4.2 which leads into the interior of a flexible electrode feed line 5 connected to the wire unit 2.2 by way of a connecting line 3. Arranged in the interior of the wire unit 2 is a balloon 6.2 which is connected to the guide wire 4.2 and which, when it is inflated, presses the wire unit against the inside wall of the vessel. In this case, the guide wire also passes through the feed line 5 which is provided with an internal lumen. It will be apparent from the Figure that the absence of an electric line within the cylindrical cross-section of the wire unit 2.2 means that guidance of the balloon is also completely unimpededly possible.

In the embodiment shown in FIG. 3—in contrast to the structure shown in FIG. 2—the guide wire 4.3 for the balloon 6.3 is not passed through the interior of the flexible electrode feed line (probe) 5, forming the feed line.

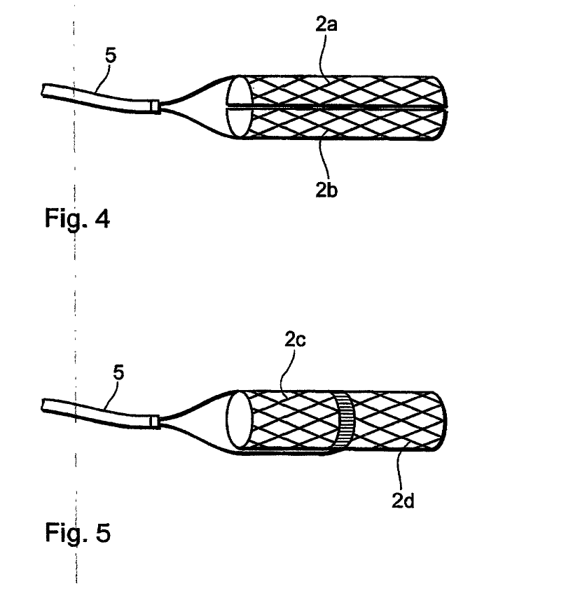

FIGS. 4 and 5 show bipolar embodiments of an electrode according to the invention, wherein in the embodiment of FIG. 4 the wire unit comprises two portions 2a, 2b which are separated from each other in the tangential direction by an insulating region while in FIG. 5 the wire unit comprises portions 2c, 2d which are insulated from each other in the axial direction. It will be seen that the structures shown in FIGS. 4 and 5 also provide that no part of the feed line is disposed in the internal cavity of the wire item. In the embodiment shown in FIG. 5 the electrical connection passes outside the portion 2c to the portion 2d. Alternatively it may also be guided in insulated relationship within the wall region of the portion 2c which is then in the form of mesh. In that case the electrical connection would then have to be provided with an insulating sheathing so that a conductive connection to the portion 2c does not exist.

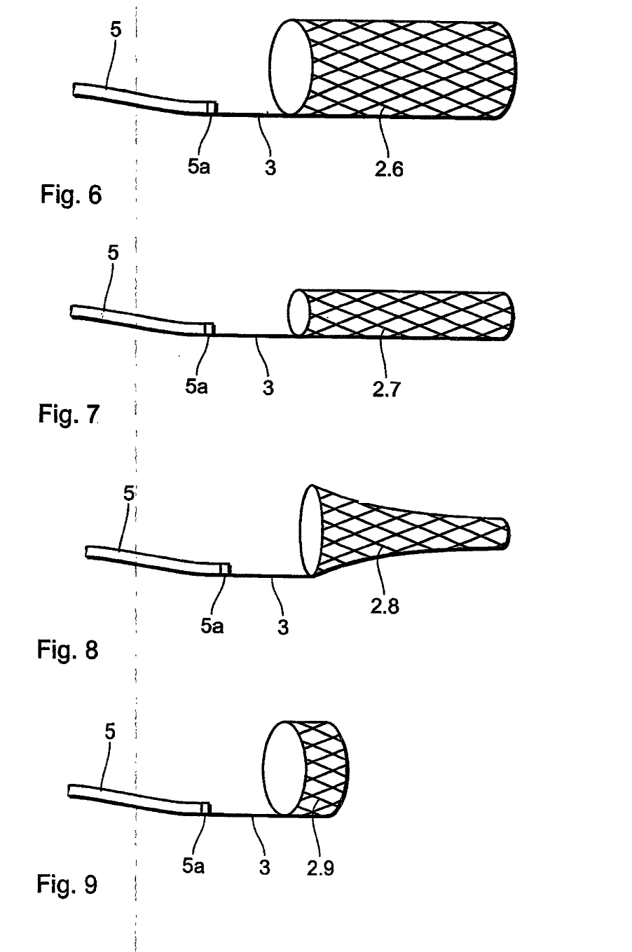

FIGS. 6 and 7 show cylindrical wire units 2.6 and 1.7 of different diameters.

FIG. 8 shows a wire unit 2.7 which is conically enlarged at one end 6.

FIG. 9 shows a narrow wire unit 2.9 in the form of a ring, of a length of about 5 mm.

FIG. 10 shows an embodiment in which the wire unit 2.10 is in the shape of a cylindrical coil so that inductive activation from the exterior is made possible thereby.

In the alternative configuration shown in FIG. 11 arranged on the guide wire 4 in the region of the interior of the coiled wire unit 2.11 is a coil 7 for producing an induction voltage. The coil 7 is fed by a control unit 10.

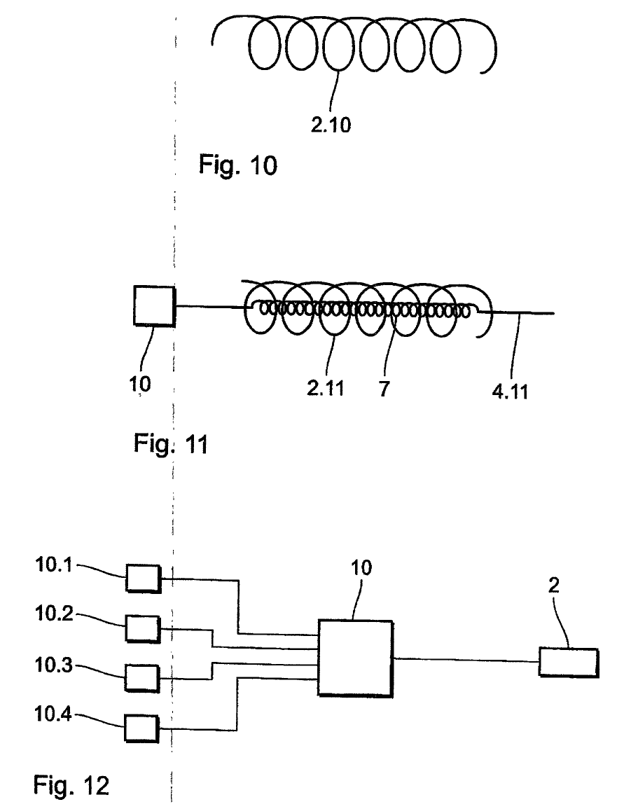

In the embodiment shown in FIG. 12 an electrode of the abovedescribed kind is provided with a stimulation control unit 10 provided with signal detectors 10.1 through 10.4 for various input signals.

Stimulation of parasympathetic autonomous nerve fibers for reducing the atrial and ventricular frequency in the case of tachycardial disrhythmia phenomena. For that purpose implantation of the wire unit forming an expansion body can be implemented in the arteria/vena jugularis interna or externa, the superior vena cava, the proximal coronary sinus or the inferior vena cava at the boundary to the right atrium.

Stimulation of autonomous nerve fibers for improving the coronary artery blood supply. For that purpose implantation of the wire unit forming an expansion body can be implemented in the arteria/vena jugularis interna or externa and in the coronary sinus.

Stimulation of sympathetic autonomous nerve fibers for the treatment of arterial hypotonia and heart pumping weakness in a case of acute and chronic heart insufficiency. For that purpose implantation of the wire unit forming an expansion body can be implemented in the arteria/vena subclavia, the pulmonary veins or the aorta.

Stimulation of sympathetic autonomous nerve fibers for the treatment of arterial hypotonia and bradycardia in the case of neuro-cardiogenic syncopes. For that purpose implantation of the wire unit forming an expansion body can be implemented in the arteria/vena subclavia, the pulmonary veins or the aorta.

Stimulation of parasympathetic autonomous nerve fibers for the treatment of tachycardial ventricular disrhythmias. For that purpose implantation of the wire unit forming an expansion body can be implemented in the coronary sinus or the pulmonary artery.

Stimulation of parasympathetic nerves which innervate the atria for preventing an atrial remodelling process. For that purpose implantation of the wire unit forming an expansion body can be implemented in the arteria/vena jugularis interna or externa, the superior vena cava or the right pulmonary artery.

Stimulation of parasympathetic nerves which innervate the atria/ventricles for a reduction in the atrial/ventricular defibrillation threshold. For that purpose implantation of the wire unit forming an expansion body can be implemented in the arteria/vena jugularis interna or external the superior vena cava or the right pulmonary artery.

Stimulation of autonomous parasympathetic nerve fibers for the treatment of cerebral convulsions (epilepsy). For that purpose implantation of the wire unit forming an expansion body can be implemented in the arteria/vena jugularis interna or externa.

Stimulation of the carotid sinus nerves for the treatment of angina pectoris complaints. For that purpose implantation of the wire unit forming an expansion body can be implemented in the arteria/vena jugularis interna or externa.

Stimulation of autonomous nerves which regulate gastro-intestinal and bladder motility and control male erection. For that purpose implantation of the wire unit forming an expansion body can be implemented in the inferior vena cava and the feeds thereto, the aorta abdominalis and the outflows therefrom (for example aa. mesentericae) or the arterial and venus iliac vessels.

High-frequency, sub-threshold electrical stimulation of the ventricular myocardium for the promotion of angiogenesis after cardiac infarcts or myocardial blood supply disturbances. For that purpose implantation of wire item electrodes can be implemented in the coronary arteries or the coronary sinus and its feeds.

In this respect the sensors 10.1 through 10.4 represent by way of example elements which correspondingly sense the state of activation for the stimulation control unit 10 and cause it to deliver a suitable control voltage or control current of appropriate form, duration and possibly frequency to the wire unit connected on the output side thereof.

1. An electrode for intravascular stimulation, cardioversion and/or defibrillation in the form of a stimulation probe which can be fixed in arterial/venous vessels of the body and by way of which electrical or magnetic pulses and defibrillation/cardioversion shocks can be delivered, which is provided with a feed line, characterised in that there is provided a metallic, electrically conductive, tubular wire unit which adjoins the feed line in the axial direction and which forms an expansion body and which can be deployed in the corresponding vessel and which under expansion bears from the interior against the wall of the vessel.

2. An electrode as set forth in claim 1 characterised in that provided as a drive means for expansion in the interior of the wire unit which is plastically deformable is a pneumatically or hydraulically inflatable balloon body.

3. An electrode as set forth in claim 1 characterised in that the wire unit is resilient and surrounded by a compression sleeve, wherein expansion of the wire body occurs automatically due to removal of a compression sleeve (self-expansion) from the wire unit in a pre-stressed compressed condition.

4. An electrode as set forth in one of claims 1 through 3 characterised in that the entire surface of the wire unit or one or more electrically mutually insulated portions thereof are in the form of a unipolar, bipolar or multipolar stimulation pole.

5. An electrode as set forth in one of claims 1 through 4 characterised in that the wire unit is in the nature of a cylindrical coil.

6. An electrode as set forth in claim 5 characterised in that the cylindrical coil includes a plurality of electrically mutually insulated coil portions.

7. An electrode as set forth in claim 5 or claim 6 characterised in that there is provided an induction unit by means of which the electrode can be inductively supplied with voltage.

8. An electrode as set forth in one of claims 1 through 7 characterised in that the diameter of the wire unit changes in the longitudinal direction.

9. An electrode as set forth in claim 8 characterised in that the wire unit is of a conical configuration for insertion into the proximal coronary sinus.

10. An electrode as set forth in one of claims 1 through 9 characterised in that the surface is coated with a medicament, in particular with a substance for preventing vessel damage.

11. An electrode as set forth in claim 7 characterised in that there is provided an induction unit by means of which the electrode is inductively heatable.

12. An electrode as set forth in one of the preceding claims characterised in that a further portion of the feed line, which portion extends in the longitudinal direction parallel at least to a portion of the wire unit, extends in the form of an insulated region within the cylindrical wall region produced by the wire unit, or outside the wire unit.

13. An electrode as set forth in one of the preceding claims characterised in that there is provided a control unit which produces control signals for one or more of the following uses and which is electrically conducted to the wire unit:
Stimulation of parasympathetic autonomous nerve fibers for reducing the atrial and ventricular frequency in the case of tachycardial disrhythmia phenomena, wherein implantation of the wire unit forming an expansion body is implemented in the arteria/vena jugularis interna or externa, the superior vena cava, the proximal coronary sinus or the inferior vena cava at the boundary to the right atrium.
Stimulation of autonomous nerve fibers for improving the coronary artery blood supply, wherein implantation of the wire unit forming an expansion body is implemented in the arteria/vena jugularis interna or externa and in the coronary sinus.
Stimulation of sympathetic autonomous nerve fibers for the treatment of arterial hypotonia and heart pumping weakness in a case of acute and chronic heart insufficiency, wherein implantation of the wire unit forming an expansion body is implemented in the arteria/vena subclavia, the pulmonary veins or the aorta.
Stimulation of sympathetic autonomous nerve fibers for the treatment of arterial hypotonia and bradycardia in the case of neuro-cardiogenic syncopes, wherein implantation of the wire unit forming an expansion body is implemented in the arteria/vena subclavia, the pulmonary veins or the aorta.
Stimulation of parasympathetic autonomous nerve fibers for the treatment of tachycardial ventricular disrhythmias, wherein implantation of the wire unit forming an expansion body is implemented in the coronary sinus or the pulmonary artery.
Stimulation of parasympathetic nerves which innervate the atria for preventing an atrial remodelling process, wherein implantation of the wire unit forming an expansion body is implemented in the arteria/vena jugularis interna or externa, the superior vena cava or the right pulmonary artery.
Stimulation of parasympathetic nerves which innervate the atria/ventricles for a reduction in the atrial/ventricular defibrillation threshold, wherein implantation of the wire unit forming an expansion body is implemented in the arteria/vena jugularis interna or externa, the superior vena cava or the right pulmonary artery.
Stimulation of autonomous parasympathetic nerve fibers for the treatment of cerebral convulsions (epilepsy), wherein implantation of the wire unit forming an expansion body is implemented in the arteria/vena jugularis interna or externa.
Stimulation of the carotid sinus nerves for the treatment of angina pectoris complaints, wherein implantation of the wire unit forming an expansion body is implemented in the arteria/vena jugularis interna or externa.
Stimulation of autonomous nerves which regulate gastrointestinal and bladder motility and control male erection, wherein implantation of the wire unit forming an expansion body is implemented in the inferior vena cava and the feeds thereto, the aorta abdominalis and the outflows therefrom or the arterial and venous iliac vessels.
High-frequency, sub-threshold electrical stimulation of the ventricular myocardium for the promotion of angiogenesis after cardiac infarcts or myocardial blood supply disturbances, wherein implantation of the wire item electrodes forming an expansion body is implemented in the coronary arteries or the coronary sinus and its feeds.
